(19) United States
(12) Patent Application Publication
(10) Pub. No.: US 2025/0064731 A1
(43) Pub. Date: Feb. 27, 2025

(54) A LIQUID PHARMACEUTICAL FORMULATION OF CLONIDINE HYDROCHLORIDE

(71) Applicant: NOVUMGEN LIMITED, London (GB)

(72) Inventor: Vishal Kumar Barot, London (GB)

(73) Assignee: NOVUMGEN LIMITED, London, GB (GB)

(21) Appl. No.: 18/729,547

(22) PCT Filed: Jan. 16, 2023
(86) PCT No.: PCT/IB2023/050355

§ 371 Date: Jul. 17, 2024

(30) Foreign Application Priority Data
Jan. 18, 2022 (GB) 2200539.1

(51) Int. Cl. A61K 9/08; A61K 9/00; A61K 31/4168; A61K 47/14; A61K 47/26

(52) U.S. Cl. CPC A61K 9/08; A61K 9/0095; A61K 31/4168; A61K 47/14; A61K 47/26

(57) ABSTRACT

The present invention is a liquid pharmaceutical formulation of clonidine hydrochloride. The present invention also a liquid pharmaceutical formulation of clonidine hydrochloride comprising clonidine hydrochloride, preservative, sweetener and a suitable vehicle, wherein the formulation is free of buffer. The present invention also a liquid pharmaceutical formulation of clonidine hydrochloride and process of preparing the same.

### FIELD OF THE INVENTION

The present invention is all about a liquid pharmaceutical formulation of clonidine hydrochloride. The present invention also relates to a liquid pharmaceutical formulation of clonidine hydrochloride comprising clonidine hydrochloride, preservative, sweetener and a suitable vehicle which is free of buffering agent. The present invention also relates to a liquid pharmaceutical formulation of clonidine hydrochloride and process of preparing the same.

### BACKGROUND OF THE INVENTION

Clonidine, N-(2,6-dichlorophenyl)-4,5-dihydro-1H-imidazol-2-amine, corresponds in structure to Formula (I) below.

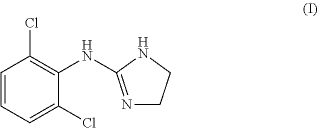 [SMILES: Clc1cccc(Cl)c1NC1=NCCN1]

Clonidine, including its hydrochloride salt, is a well known drug effective in treatment of a wide range of clinical disorders. Clonidine is particularly useful in treatment of circulatory disorders including hypertension and cardiovascular disease related thereto, congestive heart failure and cardiomyopathy.

Clonidine hydrochloride is a drug used to treat high blood pressure (hypertension). Lowering high blood pressure helps prevent strokes, heart attacks, and kidney problems.

Clonidine hydrochloride is an @2-adrenergic agonist medication used to treat high blood pressure, attention deficit hyperactivity disorder, drug withdrawal, menopausal flushing, diarrhoea, spasticity and certain pain conditions.

High blood pressure is a common condition and when not treated, can cause damage to the brain, heart, blood vessels, kidneys and other parts of the body. Damage to these organs may cause heart disease, a heart attack, heart failure, stroke, kidney failure, loss of vision, and other problems. In addition to taking medication, making lifestyle changes will also help to control your blood pressure.

These changes include eating a diet that is low in fat and salt, maintaining a healthy weight, exercising at least 30 minutes most days, not smoking, and using alcohol in moderation.

The commercially available clonidine is in the form of immediate release tablet Catapres® in the strength of 0.1 mg, 0.2 mg and 0.3 mg. Few more formulations of Clonidine are available in the market as transdermal patch (Catapres-TTS), or as an injectable form to be given epidurally, directly to the central nervous system.

Commercially available oral dosage form is administered three to four times a day or via a transdermal patch. After oral administration, clonidine is almost completely absorbed from the gastrointestinal tract and is subject to rapid liver metabolism. A peak plasma level is generally reached within 3 to 5 hours and the plasma half-life is about 12 to about 16 hours and has an elimination half-life of about 6 to about 24 hours.

Solid dosage forms are the most significant dosage forms in pharmaceuticals; it has one or more unit dose of medicament because of ease of manufacturing, storage, stability etc. However, on the other hand solid dosage form is not easy to swallow, particularly for children, and the elderly, and it cannot be given to the unconscious patient. In these types of cases solid formulation not suitable for patients.

There is higher flexibility in dosing in liquid formulation compared to solid dosage forms like tablet and capsules. The dose of the drug substance can be easily and conveniently adjusted by measuring a different volume. If given orally, liquid dosage forms are rapidly available for absorption than tablets and capsules. Liquid formulation is most suitable dosage form for patients.

There is no any oral solution available in market of clonidine hydrochloride without buffer. There is a need in the society to have heat stable and chemically stable composition of clonidine hydrochloride which can be manufactured easily with less complexity and have longer shelf life even at room temperature.

In the present invention, Inventors have found that oral solution of liquid oral solution without use of buffer can be prepared with stability and effective ness as described herein in the specification.

### OBJECTIVE OF THE INVENTION

The main objective of the present invention is to provide a liquid pharmaceutical formulation of clonidine hydrochloride without buffer.

Yet another objective of the present invention is a liquid pharmaceutical formulation of clonidine hydrochloride which is stable.

Another objective of the present invention is to provide a liquid pharmaceutical formulation of clonidine hydrochloride which is having enhanced effectiveness.

Yet another objective of the present invention is to provide a liquid pharmaceutical formulation of clonidine hydrochloride which is having improved patient compliance.

Yet another objective of this invention is to provide a liquid pharmaceutical formulation of clonidine hydrochloride which reduces number of excipients in the formulation.

### SUMMARY OF THE INVENTION

The present invention is about a liquid pharmaceutical formulation of clonidine hydrochloride without buffer.

The main aspect of the present invention is to provide a liquid pharmaceutical formulation of clonidine hydrochloride comprising clonidine hydrochloride, preservative, sweetener and a suitable vehicle, wherein the formulation is free of buffer.

Another aspect of the present invention is to provide a liquid pharmaceutical formulation of clonidine hydrochloride and process of preparing the same.

The details of one or more aspect of the invention are set forth in the description below. Other features, objects and advantages of the invention will be apparent from the description.

### DETAILED DESCRIPTION OF THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

As used herein, the term “formulation” or “composition” unless otherwise defined refers to granules and/or solid oral pharmaceutical dosage forms or solid dispersions, suspension, solution of the invention.

As used herein, whether in a transitional phase or in the body of a claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a composition, the term “comprising” means that the composition includes at least the recited features or components, but may also include additional features or components.

As used herein, the singular forms “a,” “an” and “the” specifically also encompass the plural forms of the terms to which they refer, unless the content clearly dictates otherwise.

The term “about” is used herein to means approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

The present invention overcomes the aforesaid drawbacks of the above, and other objects, features and advantages of the present invention will now be described in greater detail. Also, the following description includes various specific details and are to be regarded as merely exemplary. Accordingly, those of ordinary skill in the art will recognize that: without departing from the scope and spirit of the present disclosure and its various embodiments there may be any number of changes and modifications described herein.

As per one embodiment stable composition form of the present invention without use of buffer represents an ideal dosage form to get freedom from complex manufacturing procedure and excipients.

As per one main embodiment of the present invention the composition of present invention as described herein provide a liquid pharmaceutical formulation of clonidine hydrochloride.

As will be understood by one skilled in the art, for any and all purposes, such as in terms of providing a written description, all ranges disclosed herein also encompass any and all possible sub ranges and combinations of sub ranges thereof. Any listed range can be easily recognized as sufficiently describing and enabling the same range being broken down into at least equal halves, thirds, quarters, fifths, tenths, et cetera As a non-limiting example, each range discussed herein can be readily broken down into a lower third, middle third and upper third, et cetera As will also be understood by one skilled in the art all language such as “up to,” “at least,” and the like include the number recited and refer to ranges which can be subsequently broken down into sub ranges as discussed above.

As per preferred embodiment, the pharmaceutical composition is in the form of liquid composition for oral administration.

As used herein, the term “about” means plus or minus 10% of the numerical value of the number with which it is being used.

By “pharmaceutically acceptable”, it is meant the carrier, diluent or excipient must be compatible with the other ingredients of the topical formulation and not deleterious to the recipient thereof.

A ‘therapeutically effective amount” or “effective amount” of a composition is a predetermined amount calculated to achieve the desired effect, i.e., to induce a favourable immunological response.

The main embodiment of present invention is about a liquid pharmaceutical formulation of clonidine hydrochloride comprising clonidine hydrochloride, preservative, sweetener and a suitable vehicle, wherein the formulation is free of buffer.

### Clonidine Hydrochloride:

Clonidine is chemically known as N-(2,6-dichlorophenyl)-4,5-dihydro-IH-imidazol-2-amine and can be represented as below structure;

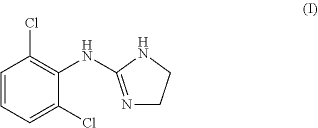 [SMILES: Clc1cccc(Cl)c1NC1=NCCN1]

Clonidine is known since very long and widely used as alpha-adrenergic agonist. Clonidine is an α₂-adrenergic receptor agonist that exhibits affinity for central presynaptic a2 receptors in the sympathetic nervous system. Clonidine is known to be effective in the treatment of a many clinical disorders including hypertension; Tourette's syndrome; prophylaxis of common migraine headaches; and decreasing hyperactivity, impulsivity and over excitability in Attention Deficit Hyperactivity Disorder, manic states and many other clinical syndromes.

As per one embodiment the Clonidine hydrochloride can be present in the composition in an amount from about 0.1 to 500 mg/5 ml, preferably in the range from about 0.1 to 450 mg/5 ml, preferably in the range from about 0.1 to 400 mg/5 ml, more preferably in the range from about 0.5 to 350 mg/5 ml, more preferably in the range from about 0.5 to 300 mg/5 ml, more preferably in the range from about 0.5 to 250 mg/5 ml, more preferably in the range from about 1 to 200 mg/5 ml, more preferably in the range from about 5 to 150 mg/5 ml, more preferably in the range from about 10 to 120 mg/5 ml, more preferably in the range from about 15 to 100 mg/5 ml, more preferably in the range from about 20 to 80 mg/5 ml, more preferably in the range from about 25 to 50 mg/5 ml or any other range in between thereof. In a most preferred embodiment, the Clonidine hydrochloride is present in the range from 1 to 10 mg/5 ml.

Vehicle for present invention can be used as a base for present invention. Vehicle can be considered as any inert substance, or mixture of substances, added to increase the volume of the composition of present invention in order to make the pharmaceutical composition of the present invention suitable form. It also plays multiple role in term of also act as solubilizer and to facilitate the solubilization and avoid precipitation.

As per one preferred embodiment the vehicle can be selected from water, sorbitol, xylitol, maltitol, lactitol, isomalt, erythritol, lauryl lactate (LL), lauryl alcohol (LA), benzyl alcohol (BA), benzyl benzoate (BB), propylene glycol, polyethyleneglycol, triglycerides (triolein, trilaurin, tricarprin, tricaprylin), ethanol, isopropanol, t-butyl alcohol, cyclohexanol, glycerin or glycerol. In the present invention, most preferably vehicle is water.

As per one embodiment of the present invention, the vehicle can be present in the composition in an amount required to make up the volume of the required batch size or quantity.

Preservatives are substances that are commonly added to pharmaceutical products in order to prolong their shelf life. The addition of preservatives to such products, especially to those that have higher water content, is essential for avoiding alteration and degradation by microorganisms during storage. A preservative is a natural or synthetic chemical added to various products which helps to prevent microbial decomposition.

As per one embodiment of the present invention preservative can be selected from Methyl parahydroxybenzoate, ethyl parahydroxybenzoate, propyl parahydroxybenzoate, Sodium Benzoate, Benzoic acid, Potassium Nitrate and Calcium Sorbate. In the present invention, most preferably preservative is Methyl parahydroxybenzoate.

As per one embodiment of the present invention, the preservative can be present in the composition in an amount from 0.1 to 1000 mg/5 ml, preferably in the range from about 0.1 to 800 mg/5 ml, preferably in the range from about 0.1 to 700 mg/5 ml, more preferably in the range from about 1 to 300 mg/5 ml, more preferably in the range from about 1 to 100 mg/5 ml or any other range in between thereof. In a most preferred embodiment, the preservative is present in the range from 1 to 50 mg/5 ml.

Sweetening agents are employed in liquid formulations designed for oral administration specifically to increase the palatability of the therapeutic agent.

As per one embodiment of the present invention, the sweetening agent is selected from sucralose, cyclamate, saccharin, saccharin sodium, molasses, stevia and erythritol. In the present invention, most preferably sweetener is sucralose.

As per one embodiment of the present invention, the sweetener can be present in the composition in an amount from 0.1 to 1000 mg/5 ml, preferably in the range from about 0.1 to 800 mg/5 ml, preferably in the range from about 0.1 to 700 mg/5 ml, more preferably in the range from about 1 to 300 mg/5 ml, more preferably in the range from about 1 to 100 mg/5 ml or any other range in between thereof. In a most preferred embodiment, the preservative is present in the range from 1 to 20 mg/5 ml.

As per one embodiment, the present invention relates to process for preparing a liquid pharmaceutical formulation of clonidine hydrochloride. Particularly, a process for the preparation of a stable liquid pharmaceutical composition of clonidine hydrochloride.

As per one preferred embodiment, the process of preparing a liquid pharmaceutical formulation of clonidine hydrochloride comprising the steps:
a) weighing of clonidine hydrochloride, methyl parahydroxybenzoate, sucralose and purified water and transferring in a vessel;
b) adding methyl parahydroxybenzoate to step (a) under temperature between 90° C.-95° C. and stirring the solution until all the contents in the vessel are dissolved;
c) making up the volume to 40 liters with purified water and cooling the solution to room temperature;
d) transferring sucralose to step (c) solution and mixing for 5 to 7 minutes;
e) adding clonidine hydrochloride into step (d) solution and mixing for 10 to 15 minutes;
f) making up the volume of step (e) solution with sufficient quantity of purified water and mixing for 8 to 12 minutes;
g) keeping aside the step (f) solution in mixing tank and allow the solution to become clear and colourless;
h) checking the pH of step (g) solution between 5.5 to 7.5 and filtering through 125 micron sieve;
i) filling the solution of step (h) into type III amber glass bottles.

As per one embodiment buffers are usually used to maintain the specific pH value. Buffers ensure the stability and solubility of the drug. However present invention provides liquid formulation which is made without buffer and also stable and effective.

As per one preferred embodiment the advantages of the present invention is chemically stable liquid pharmaceutical composition of clonidine hydrochloride which is oral formulation and ready to use. Liquid formulation are rapidly available for absorption than tablets and capsules. Another advantage of liquid dosage form is that it provides the maximum therapeutic response in patients who have problem of swallowing solid dosage forms and/or to produce rapid therapeutic effects.

As per one embodiment, the present invention is about liquid pharmaceutical formulation of clonidine hydrochloride comprising 1 to 100 mg/5l of clonidine hydrochloride, 1 to 50 mg/5 ml of preservative, 1 to 100 mg/5 ml of sweetener and a vehicle, wherein the formulation is free of buffer.

As per another embodiment, the present invention is about liquid pharmaceutical formulation of clonidine hydrochloride comprising 1 to 50 mg/5l of clonidine hydrochloride, 1 to 20 mg/5 ml of preservative, 1 to 50 mg/5 ml of sweetener and a vehicle, wherein the formulation is free of buffer.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLES

### Example 1: A Liquid Pharmaceutical Formulation of Clonidine Hydrochloride

TABLE 1
Liquid formulation of clonidine hydrochloride

| Ingredients | Quantity (mg/5 ml) |
|---|---|
| Clonidine hydrochloride | 0.05 |
| Methyl parahydroxy benzoate | 9.00 |
| Sucralose | 2.00 |
| Purified Water | Up to 5 ml |

Procedure:
a) clonidine hydrochloride, methyl parahydroxybenzoate, sucralose and purified water were weigh up and transferred in a vessel;
b) methyl parahydroxybenzoate was added to step (a) under temperature between 90° C.-95° C. and the solution was stirred until all the contents in the vessel are dissolved;
c) the volume was made up to 40 litres with purified water and cooled down the solution to room temperature;
d) sucralose was transferred to step (c) solution and mixed for 5 to 7 minutes;
e) clonidine hydrochloride was added into step (d) solution and mixed for 10 to 15 minutes;
f) the volume of step (e) solution was made up with sufficient quantity of purified water and mixed for 8 to 12 minutes;
g) the step (f) solution kept aside in mixing tank and the solution was allowed to become clear and colourless;
h) the pH of step (g) solution was checked between 5.5 to 7.5 and filtered through 125 micron sieve;
i) the solution of step (h) was filled into type III amber glass bottles.
j) the filled bottles were packed using current approved packaging specifications.

### Example 2: Stability Study

The stability of the final composition was performed. The stability were carried out at 2 temperatures which is 25° C.±2° C. and 40° C.±2° C. at Initially and after 1 month.

The following table shows the result:

| Storage Condition | | | 40° C. ± 2° C. | | 25° C. ± 2° C. | |
|---|---|---|---|---|---|---|
| Test | Specification | Initial | 1 month | 3 month | 1 month | 3 month |
| Description | A clear | Complies | Complies | Complies | Complies | Complies |
| | colorless or | | | | | |
| | almost | | | | | |
| | colorless | | | | | |
| | solution | | | | | |
| pH | Limit to be | 6.21 | 6.31 | 6.15 | 6.32 | 6.27 |
| | established | | | | | |
| Assay | 95.0%- | 100.4 | 99.8 | 102.1 | 99.0 | 104.8 |
| | 105.0% of | | | | | |
| | label claim | | | | | |
| | of Clonidine | | | | | |
| | HCl | | | | | |

Related Substances

| | | | | | | |
|---|---|---|---|---|---|---|
| Single | NMT 1.0% | ND | ND | ND | ND | ND |
| maximum | | | | | | |
| unknown | | | | | | |
| impurity | | | | | | |
| Total | NMT 2.0% | ND | ND | ND | ND | ND |
| Impurity | | | | | | |

We claim:

1. A liquid pharmaceutical formulation of clonidine hydrochloride comprising clonidine hydrochloride, preservative, sweetener and a vehicle, wherein the formulation is free of buffer.

2. The liquid pharmaceutical formulation of clonidine hydrochloride as claimed in claim 1, wherein the preservative is selected from methyl parahydroxybenzoate, ethyl parahydroxybenzoate, propyl parahydroxybenzoate, Sodium Benzoate, Benzoic acid, Potassium Nitrate and Calcium Sorbate.

3. The liquid pharmaceutical formulation of clonidine hydrochloride as claimed in claim 1, wherein the sweetener is selected from sucralose, cyclamate, saccharin, saccharin sodium, molasses, Stevia and Erythritol.

4. The liquid pharmaceutical formulation of clonidine hydrochloride as claimed in claim 1, wherein the vehicle is selected from the water, sorbitol, xylitol, maltitol, lactitol, isomalt, erythritol, lauryl lactate (LL), lauryl alcohol (LA), benzyl alcohol (BA), benzyl benzoate (BB), propylene glycol, polyethyleneglycol, triglycerides (triolein, trilaurin, tricarprin, tricaprylin), ethanol, isopropanol, t-butyl alcohol, cyclohexanol, glycerin or glycerol.

5. The liquid pharmaceutical formulation of clonidine hydrochloride as claimed in claim 1, wherein the clonidine hydrochloride present in the composition is in the range from 1 to 100 mg/5 ml.

6. The liquid pharmaceutical formulation of clonidine hydrochloride as claimed in claim 1, wherein the preservative present in the composition is in the range from 1 to 50 mg/5 ml.

7. The liquid pharmaceutical formulation of clonidine hydrochloride as claimed in claim 1, wherein the sweetener present in the composition is in the range from 1 to 100 mg/5 ml.

8. The liquid pharmaceutical formulation of clonidine hydrochloride as claimed in claim 1, wherein the process of preparation of liquid formulation of clonidine hydrochloride comprises the steps of;
a) weighing of clonidine hydrochloride, methyl parahydroxybenzoate, sucralose and purified water and transferring in a vessel;
b) adding methyl parahydroxybenzoate to step (a) under temperature between 90° C.-95° C. and stirring the solution until all the contents in the vessel are dissolved;
c) making up the volume to 40 liters with purified water and cooling the solution to room temperature;
d) transferring sucralose to step (c) solution and mixing for 5 to 7 minutes;
e) adding clonidine hydrochloride into step (d) solution and mixing for 10 to 15 minutes;
f) making up the volume of step (e) solution with sufficient quantity of purified water and mixing for 8 to 12 minutes;
g) keeping aside the step (f) solution in mixing tank and allow the solution to become clear and colourless;
h) checking the pH of step (g) solution between 5.5 to 7.5 and filtering through 125 micron sieve;
i) filling the solution of step (h) into type III amber glass bottles.
